(19) United States
(12) Patent Application Publication
(10) Pub. No.: US 2012/0271064 A1
(43) Pub. Date: Oct. 25, 2012

(54) METHOD FOR PRODUCING HYDROXYALKYL(METH)ACRYLATES

(75) Inventors: Björn Henninger, Koln (DE); Ursula Tracht, Leverkusen (DE); Sigurd Buchholz, Koln (DE); Claudia Willems, Leverkusen (DE); Rolf Bachmann, Bergisch Gladbach (DE); Michael Ludewig, Odenthal (DE); Wolfgang Fischer, Meerbusch (DE)

(73) Assignees: BAYER MATERIAL SCIENCE AG, Leverkusen (DE); BAYER TECHNOLOGY SERVICES GMBH, Leverkusen (DE)

(21) Appl. No.: 13/378,585

(22) PCT Filed: Jul. 6, 2010
(86) PCT No.: PCT/EP2010/004115

§ 371 Date: Dec. 27, 2011

(30) Foreign Application Priority Data
Jul. 18, 2009 (DE) 10 2009 033 831.4

(51) Int. Cl. C07C 67/29; C07C 69/54

(52) U.S. Cl. 560/224

(57) ABSTRACT

The present invention relates to a continuous process for preparing hydroxyalkyl (meth)acrylates, more particularly those hydroxyalkyl (meth)acrylates which have more than one (meth)acrylate group per molecule.

The present invention relates to a continuous process for preparing hydroxyalkyl (meth)acrylates, more particularly those hydroxyalkyl (meth)acrylates which have more than one (meth)acrylate group per molecule.

Hydroxyalkyl (meth)acrylates are known. Their uses include reaction with isocyanate-containing compounds for preparing urethane (meth)acrylates and unsaturated polyurethane dispersions (see e.g. EP1700873A1).

In particular they are ingredients of coating compositions which are cured by radical polymerization (see e.g. EP1541609A2).

Hydroxyalkyl (meth)acrylates here and below are specific esters of acrylic acid or of methacrylic acid, with the general formula (1):

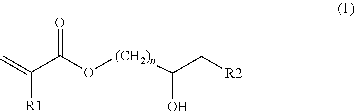

In this formula, R1 =H or CH₃ and n is an integer (1, 2, 3, . . . ). R2 is any desired group attached preferably via a nitrogen or oxygen atom, e.g. alkoxy-, alkenoxy-, alkynoxy-, phenoxy-, amino-, carboxy-, acryloyloxy-, methacryloyloxy- et cetera.

One specific representative of the hydroxyalkyl (meth)acrylates is 3-acryloyloxy-2-hydroxypropyl methacrylate, also identified below as GAMA:

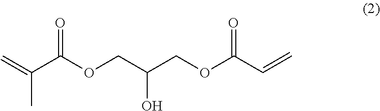

It is known that compounds containing acrylate and/or methacrylate groups are temperature-sensitive and/or shear-sensitive and that spontaneous polymerization may occur as a result of mechanical and/or thermal exposure (see e.g. EP 1 293 547 B1).

The exothermic nature of the polymerization may lead to the incidence of what are called hot spots, which when preparing hydroxyalkyl (meth)acrylates leads at best to products which are not uniform and not reproducible, and at worst may cause reaction runaway. Particularly in the case of hydroxyalkyl (meth)acrylates wherein the group R2 in the structural formula (1) comprises a further acrylate or methacrylate function, the hazard potential in the case of batchwise production is particularly high, since in such compounds there is a high concentration of temperature-sensitive and/or shear-sensitive groups. One representative of these particularly hazardous compounds is GAMA. The preparation of such compounds, accordingly, imposes very exacting requirements on safety. According to the prior art, compounds of this kind are prepared under precisely controlled conditions in order to prevent the incidence of hot spots. The temperature is held well below the temperature at which hydroxyalkyl (meth)acrylates may undergo follow-on reactions, such as spontaneous polymerization. The low temperatures result in long reaction times and hence in a poor space-time yield. The reaction batches are selected to be correspondingly small, in order to minimize the hazard to the environment in the event of unwanted follow-on reactions. The amounts converted in batch processes in accordance with the prior art are, correspondingly, low.

EP-A1693359, for example, describes a batch process for preparing GAMA in which glycidyl methacrylate is reacted with acrylic acid at a temperature of 80° C. by catalysis with weakly Lewis-acidic borane compounds such as trisdimethylaminoborane, for example, to form GAMA. The reaction times amount to 24 to 48 hours or more.

In this respect the synthesis of hydroxyalkyl (meth)acrylates is subject to a series of requirements which run counter to rapid and uncomplicated preparation. This is true in particular of those hydroxyalkyl (meth)acrylates of the structural formula (1) in which R2 comprises an acrylate or methacrylate function. It would be desirable to be able to carry out the synthesis of hydroxyalkyl (meth)acrylates at higher temperatures than described in the prior art, in order to shorten the reaction times. Here, however, there is a risk of follow-on reactions occurring, particularly the radical polymerization of the unsaturated double bond in the (meth)acrylate. This is true particularly of hydroxyalkyl (meth)acrylates of the structural formula (1) in which R2 comprises a further acrylate or methacrylate function.

On the basis of the prior art, therefore, the problem which exists is that of providing a process for preparing hydroxyalkyl (meth)acrylates that allows a higher space-time yield with comparable product quality than the processes described in the prior art. The process in particular is to allow the preparation of hydroxyalkyl (meth)acrylates which as group R2 in the structural formula (1) have an acrylate or methacrylate function.

The present invention accordingly provides a process for preparing hydroxyalkyl (meth)acrylates, characterized in that at least one compound A and at least one compound B are commingled continuously in a reaction apparatus and conveyed in the form of a reaction mixture at a temperature from +20° C. to +200° C. through the reaction apparatus, the at least one compound A having at least one epoxide group, the at least one compound B having at least one nucleophilic group capable under nucleophilic attack of opening an epoxide group, and A and/or B having at least one (meth)acrylate group.

Continuous reactions in the sense of the invention are those in which the introduction of the reactants into the reactor and the removal of the products from the reactor take place simultaneously but at separate locations, whereas, in the case of discontinuous reaction, the reaction steps involving introduction of the reactants, chemical reaction, and removal of the products take place one after another. The continuous procedure is economically advantageous, since it avoids reactor downtimes due to filling and emptying operations, and avoids long reaction times due to safety provisions, reactor-specific heat-exchange procedures, and heating and cooling operations of the kind which occur in batch processes.

The process of the invention is characterized in that at least one compound A and at least one compound B are commingled continuously in a reaction apparatus and are conveyed as a reaction mixture through the reaction apparatus. Along the residence path through the reaction apparatus, there is continuous reaction of A and B to form a hydroxyalkyl (meth)acrylate as per structural formula (1).

The continuous reaction takes place under pressure of 0-30 bar, preferably of 0-10 bar, more preferably in the range of 0-4 bar, and at temperatures from +20° C. to +200° C., preferably in the range from +80° C. to +160° C. and more preferably in the range from +90° C. to +120° C.

Besides the compounds A and B, further components may be present in the reaction mixture or supplied thereto along the reaction section. The further components may comprise, for example, one or more compounds A and/or B, solvents and/or catalyst.

The metering rates of all the components are dependent primarily on the desired residence times and the conversions to be achieved. The higher the maximum reaction temperature, the shorter the residence time ought to be. In general, in the reaction zone, the reactants have residence times in the range from 20 seconds (20 sec) to 400 minutes (400 min), preferably in the range from 40 min to 400 min, very preferably in the range from 90 min to 300 min.

The residence time may be controlled, for example, through the volume flow rates and the volume of the reaction zone. The course of the reaction is advantageously monitored by means of various measurement installations. Particularly suitable for this purpose are installations for measuring the temperature, the viscosity, the thermal conductivity and/or the refractive index in flowing media and/or for measuring infrared and/or near-infrared spectra.

The components may be metered in separate streams to the reactor. Where there are more than two streams, they may also be supplied in bundled form. It is possible to supply streams in different proportions at different locations of the reactor, in order thus to set concentration gradients specifically, in order, for example, to bring about complete reaction. The entry point of the streams may be varied in sequence and staggered in time. For the preliminary reaction and/or for completion of the reaction, two or more reactors may also be combined.

Prior to commingling, the streams may be heated by a heat exchanger, i.e. to a temperature of −20° C. to +200° C., preferably +10° C. to +140° C., more preferably +20° C. to +120° C.

The components, more particularly the compounds A and B, are commingled preferably using mixing elements which bring about intense mixing of the reactants. It is advantageous to use an intensive mixer (t mixer) with which the reaction solutions are mixed very quickly with one another, preventing a possible radial concentration gradient. The use of microreactors/micro-mixers results in reduced shearing of the reaction mixture, and this, in the case of the shear-sensitive (meth)acrylates, results in a more secure process regime and, moreover, implies an increased product quality.

After the reactants have been commingled/mixed, they are conveyed through the reaction apparatus, which may comprise further mixing elements. Further mixing elements along the reaction section result in a preferred narrower residence-time distribution. The reaction apparatus is characterized in that it provides a residence volume in the volume from 20 seconds (20 sec) to 400 minutes (400 min), preferably in the range from 40 min to 400 min, very preferably in the range from 90 min to 300 min.

The reaction sections for use in accordance with the invention are additionally notable for their high heat transfer capacity, as characterized by the specific heat transfer rate in W/(K·m³), in other words heat transfer per kelvin of temperature difference with respect to the heat transfer medium, based on the free volume of the reactor. The reaction sections for use in accordance with the invention are characterized in that they allow a heat transfer rate of 10 to 750 kW/(K·m³), preferably 50 to 750 kW/(K·m³) and more preferably 100 to 750 kW/(K·m³).

These high heat transfer rates have the effect in particular of minimizing temperature differences between the reactor contents and the cooling medium, allowing very narrow temperature control, which is beneficial to the stability of the process and also in respect of potential formation of deposits on the surfaces.

The reaction of the starting materials takes place preferably in microstructured mixers in combination with intensive heat exchangers, which allow a narrow residence time as well as efficient temperature control. As a result of this strict process control regime, a reaction temperature is made possible which is significantly higher than in the existing process, hence allowing a drastic reduction in residence time to be realized. This is surprising in particular since the reaction temperatures used are already in the range of an exothermic product follow-on reaction that can be determined by means of differential thermal analysis (DTA).

Examples of suitable reaction apparatus are intensive heat exchangers, such as CSE-XR models from Fluitec, for example. Likewise conceivable are associations of microreactors with other heat exchangers having a greater structuring, such as exchangers from Fluitec or Sulzer, for example. A key feature in the case of these associations is the disposition of the individual reactor types in accordance with the anticipated, necessary heat output of each individual apparatus, taking account of the viscosities and pressure losses that occur.

Also appropriate is the use of the microreaction technology (u-reaction technology) using microreactors. The term “microreactor” used is a representative term for microstructured reactors which preferably operate continuously and which are known under the designation microreactor, minireactor, micro-heat exchanger, minimixer or micromixer. Examples are microreactors, micro-heat exchangers, T and Y mixers, and also micromixers from a wide variety of different companies (e.g. Ehrfeld Mikrotechnik BTS GmbH, Institut für Mikrotechnik Mainz GmbH, Siemens A G, CPC-Cellular Process Chemistry Systems GmbH, and others), as are general knowledge to the skilled person, and a “microreactor” in the sense of the present invention typically has characteristic/defining internal dimensions of up to 1 mm and may contain static mixing internals.

In addition to the heat transfer properties of the reaction section, a narrow residence-time distribution in the reactor system is likewise an advantage, allowing the residence volume that is necessary for the desired conversion to be minimized. This is typically achieved through the use of static mixing elements or of microreactors, as described above. This requirement is also typically met to a sufficient extent by intensive heat exchangers such as the CSE-XR model, for example.

It is conceivable to connect two or more reactors in series. Each of these reactors is advantageously provided with a cooling and/or heating means, such as a jacket through which a temperature-conditioned heat transfer fluid is passed.

The use of two or more, independently temperature-conditionable heating/cooling zones makes it possible, for example, to cool the flowing reaction mixture at the beginning of the reaction, in other words shortly after mixing, and to remove heat of reaction that is produced, and to heat the mixture toward the end of the reaction, in other words shortly before its removal from the reactor, in order to maximize conversion. The cooling and heating media temperature may be between +25 and +250° C., preferably below +200° C. As well as by heating and/or cooling, the temperature of the reaction mixture is also influenced by the heat of reaction. In the presence of ethylenically unsaturated compounds it is useful not to exceed particular upper temperature limits, since otherwise there is an increased risk of unwanted polymerization. For unsaturated acrylates, the maximum reaction temperature ought not to exceed levels of +250° C. It is preferred not to exceed +200° C.

In contrast to the existing semi-batch or batch processes, the continuous process of the invention allows reliable and product-compatible preparation with a significantly higher space-time yield and with reduced hold-up in the plant. From the standpoint of safety, in particular, the process of the invention allows the production of hydroxyalkyl (meth)acrylates on the larger scale as well, since the continuous process means that the hold-up in the reactor can be significantly reduced.

The process of the invention is characterized in that at least one compound A is reacted continuously with at least one compound B, the at least one compound A having at least one epoxide group, the at least one compound B having at least one nucleophilic group capable under nucleophilic attack of opening an epoxide group, and A and/or B having at least one (meth)acrylate group.

The at least one compound A and the at least one compound B preferably each comprise at least one (meth)acrylate group.

Suitable compounds A are monoepoxide compounds and also polyfunctional epoxides, more particularly difunctional or trifunctional epoxides. Examples include epoxidized olefins, glycidyl ethers of (cyclo)aliphatic or aromatic polyols, and/or glycidyl esters of saturated or unsaturated carboxylic acids. Examples of particularly suitable monoepoxide compounds include glycidyl acrylate, glycidyl methacrylate, Versatic acid glycidyl esters, butyl glycidyl ether, 2-ethylhexyl glycidyl ether, phenyl glycidyl ether, o-cresyl glycidyl ether or 1,2-epoxybutane.

Particularly suitable polyepoxide compounds are polyglycidyl compounds of the bisphenol A or bisphenol F type and also their perhydrogenated derivatives, or glycidyl ethers of polyfunctional alcohols such as butanediol, hexanediol, cyclohexanedimethanol, glycerol, trimethylolpropane or pentaerythritol.

It is likewise possible to use epoxy-functional polymers of vinyl monomers, such as monofunctional acrylates, methacrylates or styrene, for example, with proportional use of glycidyl methacrylate, for example.

Examples of suitable compounds B include carboxylic acids having a functionality of one, two or higher. Monocarboxylic acids contemplated are saturated and preferably unsaturated carboxylic acids such as benzoic acid, cyclohexanecarboxylic acid, 2-ethylhexanoic acid, caproic acid, caprylic acid, capric acid, lauric acid, natural and synthetic fatty acids, especially acrylic acid, methacrylic acid, dimeric acrylic acid or crotonic acid. Suitable dicarboxylic acids are phthalic acid, isophthalic acid, tetrahydrophthalic acid, hexahydrophthalic acid, cyclohexanedicarboxylic acid, maleic acid, fumaric acid, malonic acid, succinic acid, glutaric acid, adipic acid, azelaic acid, pimelic acid, suberic acid, sebacic acid, dodecanedioic acid, and hydrogenated dimer fatty acids.

The dicarboxylic acids can be used in the form—where available—of their anhydrides, with addition of a corresponding amount of water. Besides the pure acids, it is also possible to employ acid-functional polyesters or corresponding reaction mixtures which have been prepared with an excess of acid. Such mixtures, especially containing polyether acrylates and/or polyester acrylates with, for example, excess acrylic acid, are described in EP-A 0 976 716, EP-A 0 054 105 and EP-A 0 126 341, for example.

It is likewise possible to use acid-functional polymers, examples being polyacrylates of vinyl monomers such as, for example, monofunctional acrylates, methacrylates or styrene, with proportional use of acrylic acid or methacrylic acid, for example.

The equivalents ratio of acid to epoxide may be varied within wide ranges. Preference, however, is given to an equivalents ratio of 1.2:1.0 to 1.0:1.2, more particularly 1.05:1.00 to 1.00:1.05.

In one preferred embodiment of the process of the invention, the reaction takes place of acrylic acid, methacrylic acid and/or dimeric acrylic acid with glycidyl acrylate and/or glycidyl methacrylate, particular preference being given to a reaction of glycidyl methacrylate with acrylic acid. The reaction of the acid with the glycidyl compound takes place in an equivalents ratio of 0.90:1.00 to 1.30:1.00, preferably of 1.01:1.00 to 1.20:1.00. It may in particular be useful to use a slight excess of one component, in order to obtain particularly low residual levels of the other component in the process product. For example, with the process of the invention, given appropriate choice of the equivalents ratios, residual levels of acrylic acid or glycidyl methacrylate of below 0.1% by weight can be reliably realized.

The reaction is preferably carried out with catalysis. Catalysts contemplated are those compounds known in the literature as catalysts of the reaction of glycidyl compounds with carboxylic acids, such as, for example, tertiary amines, tertiary phosphines, ammonium compounds or phosphonium compounds, thiodiglycol, and compounds of tin, of chromium, of potassium and of caesium. Preference is given to those which are free of amine compounds or ammonium compounds. Triphenylphosphine is especially preferred.

The reaction is carried out preferably in the presence of stabilizers for acrylates and methacrylates. 15 As well as oxygen-containing gas, chemical stabilizers are suitable for avoiding premature polymerization, in an amount of 0.001-1% by weight, preferably 0.005-0.05% by weight, based on the amount of the unsaturated compounds. Stabilizers of this kind are described in, for example, Houben-Weyl, Methoden der organischen Chemie, 4th Edition, Volume XIV/1, Georg Thieme Verlag, Stuttgart 1961, page 433 ff. Examples include the following: sodium dithionite, sodium hydrogensulphide, sulphur, hydrazine, phenylhydrazine, hydrazobenzene, N-phenyl-β-naphthylamine, N-phenylethanoldiamine, dinitrobenzene, picric acid, p-nitrosodimethylaniline, diphenylnitrosamine, phenols, such as para-methoxyphenol, 2,5-di-tert-butylhydroquinone, 2,6-di-tert-butyl-4-methylphenol, p-tert-butylpyrocatechol or 2,5-di-tert-amylhydroquinone, tetramethylthiuram disulphide, 2-mercaptobenzothiazole, dimethyldithiocarbamic acid sodium salt, phenothiazine, N-oxyl compounds such as, for example, 2,2,6,6-tetramethylpiperidine N-oxide (TEMPO) or one of its derivatives. Preference is given to 2,6-di-tert-butyl-4-methylphenol and para-methoxyphenol and to mixtures thereof.

In one preferred embodiment, the process of the invention takes place with exclusion of oxygen (anaerobic conditions), using a stabilizer such as phenothiazine, for example.

Stabilizers such as phenothiazine may give rise to slight colouration. In another preferred embodiment, the process of the invention takes place using oxygen as stabilizer, which can be injected into the reaction mixture preferably via a membrane. In place of pure oxygen, it is also possible to use gas mixtures such as air, for example.

The reaction may be carried out in the presence of an organic solvent which is inert towards reactants and products and which is preferably also inert towards isocyanates. Examples are paint solvents such as butyl acetate, solvent naphtha, methoxypropyl acetate or hydrocarbons such as cyclohexane, methylcyclohexane or isooctane.

The hydroxyalkyl (meth)acrylates formed may be subjected immediately to further reaction, for example with isocyanate-containing compounds, for the purpose of preparing urethane (meth)acrylates and unsaturated polyurethane dispersions, or may first be stored or transported. Further reaction takes place preferably without additional purification, such as extraction or distillation with isocyanate-containing compounds, for example.

The invention also provides for the use of the hydroxyalkyl (meth)acrylates prepared by the process of the invention as a component in compositions curable with actinic radiation, and in the synthesis of components for compositions curable with actinic radiation.

The hydroxyalkyl (meth)acrylates prepared by means of processes of the invention are suitable in particular for preparing binders curable by radical polymerization, for—for example—paints, adhesives, sealants and others.

The invention is elucidated below by means of examples, but without being confined to these examples.

### EXAMPLE 1

### Apparatus for Implementing the Process of the Invention

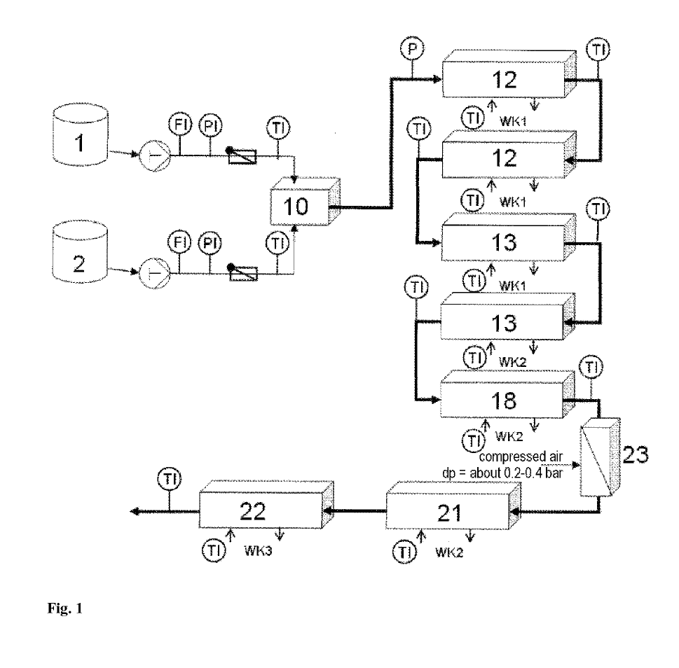

FIG. 1 shows, schematically, a construction for the implementation of the process of the invention. There are two reservoirs 1 and 2, from which the reactants can be supplied separately to the reactor. In one of the reservoirs there is preferably a compound A which has an epoxide group, and in the other reservoir there is a compound B which has a nucleophilic group. A and/or B have at least one (meth)acrylate group. Preferably, both A and B have a (meth)acrylate group.

In the present example, the reservoirs used are glass vessels having a capacity of 5 l.

The reactants are commingled in a mixer 10. In the present example, a membrane piston pump (Lewa ecodos 6S1×3) is used for each metered stream. The mixer is a cascade mixer from Ehrfeld Mikrotechnik BTS GmbH.

After the reactants have been mixed, the reaction mixture is passed through a reaction section, which in the present example is formed by 5 Fluitec heat exchangers of type CSE-XR, the heat exchangers 12 (DN25) having a volume each of about 0.37 l, the heat exchangers 13 (DN50) a volume each of about 1.7 l, and the heat exchanger 18 (DN80) a volume of about 4 l.

The serially connected heat exchangers are followed in the present example by a tube reactor 21 (DN100) having a volume of about 8 l, which is fitted with static mixing elements. The temperature conditioning of the reaction section is accomplished by means of two circuits, which are each connected in parallel and are thermally conditioned by means of thermostats (1× Huber (WK1), 1× Lauda (WK2)). The tube reactor 21 is followed by an IKSM tube reactor as after cooler, with a water cooling system WK3.

Located between reactors 18 and 21 is a gasifying installation consisting of a ceramic membrane of type Inopor nano (TiO₂, 0.9 nm, cut-off limit 450D)—through which the reaction medium flows—and a surrounding gas space, to which compressed air is supplied. The pressure on the gas side is set at about 0.2-0.4 bar higher than the pressure in the interior of the membrane. The gasifying installation is operated below its bubble-forming point, i.e. there is no gas phase formed on the side of the reaction medium.

### Example 2

### Synthesis of 3-acryloyloxy-2-hydroxypropyl methacrylate (GAMA)

The apparatus from Example 1 is used. All of the chemicals used are available commercially, from Sigma Aldrich, for example.

Reservoir 1 is charged with a GMA solution whose composition is as follows:

Glycidyl methacrylate (GMA): 98.2% by weight

Triphenylphosphine (TPP): 1.5% by weight

Phenothiazine: 0.004% by weight

Di-tert-butylmethylphenol (inhibitor KB) 0.22% by weight

Reservoir 2 is charged with acrylic acid.

The reaction apparatus is heated to 80° C. empty. Reactant is metered in from reservoir 1 with a mass flow rate of 3.07 kg/h; from reservoir 2, reactant is metered in with a mass flow rate of 1.56 kg/h.

The reactors are each thermally conditioned with a mass flow rate of 500 kg of thermostat oil (silicone oil) per hour (WK1, WK2).

After the start of the metered feeds, the plant is slowly flooded. When the reactors of the first heating circuit (WK1) have been filled, the temperature in this circuit is slowly raised, in a number of steps, to a jacket temperature of 110° C. The same procedure at the same rate is carried out with the reactors of the second thermal conditioning circuit (WK2) when they are filled, the jacket temperature set here being 110° C. After a further 3 residence times, the product (GAMA) is obtained.

Result: residual monomer content: 0.5% by weight acrylic acid, 0.48% by weight GMA

1. Process for preparing hydroxyalkyl (meth)acrylates, wherein at least one compound A and at least one compound B are commingled continuously in a reaction apparatus and conveyed in the form of a reaction mixture at a temperature from +20° C. to +200° C. through the reaction apparatus, the at least one compound A having at least one epoxide group, the at least one compound B having at least one nucleophilic group capable under nucleophilic attack of opening an epoxide group, and A and/or B having at least one (meth)acrylate group.

2. Process according to claim 1, wherein the temperature is in the range from +80° C. to +160° C.

3. Process according to claim 1 wherein the commingling of the compounds A and B takes place using a static mixer.

4. Process according to claim 1, wherein the reaction apparatus comprises further mixing elements for obtaining a narrow residence-time distribution along the reaction section.

5. Process according to claim 1, wherein the reaction apparatus has a heat transfer rate of 10 to 750 kW/(K·m³).

6. Process according to claim 1, wherein the hydroxyalkyl (meth)acrylate is a hydroxyalkyl (meth)acrylate having a structure of the formula (1) 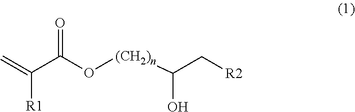
where R1=H or CH₃,
R2=alkoxy-, alkenoxy-, alkynoxy-, phenoxy-, amino-, carboxy-, acryloyloxy-, methacryloyloxy- and
n is an integer (1, 2, 3, . . . ).

7. Process according to claim 6, wherein the group R2 comprises an acrylate or methacrylate group.

8. Process according to claim 1, wherein acrylic acid, methacrylic acid and/or dimeric acrylic acid are reacted with glycidyl acrylate and/or glycidyl methacrylate.

9. Process according to claim 8, wherein the reaction of the acid with the glycidyl compound takes place in an equivalents ratio of 0.90:1.00 to 1.30:1.00.

10. Binders curable curable by radical polymerization, comprising hydroxyalkyl (meth)acrylates prepared by the process of claim 1.

11. Process of claim 2, wherein said temperature is in the range of from +90° C. to +120° C.

12. Process of claim 3 wherein said static mixer is a μ mixer.

13. Process of claim 5 wherein said heat transfer rate is 50 to 750 kW/(K·m³).

14. Process of claim 13 wherein said heat transfer rate is 100 to 750 kW/(K·m³).

15. Process of claim 9, wherein said ratio is 1.01:1.00 to 1.20:1.00
